(19) United States
(12) Patent Application Publication
(10) Pub. No.: US 2009/0053208 A1
(43) Pub. Date: Feb. 26, 2009

(54) Methods and Systems for Improving Tissue Perfusion

(75) Inventor: ASHA NAYAK, Sunnyvale, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/194,957

(22) Filed: Aug. 20, 2008

Related U.S. Application Data
(60) Provisional application No. 60/956,754, filed on Aug. 20, 2007.

(51) Int. Cl. A61K 39/395; C12N 5/06; A61K 35/14; A61K 38/48; A61P 9/00; A61K 38/43

(52) U.S. Cl. 424/130.1; 435/325; 424/532; 424/94.1; 424/94.64

(57) ABSTRACT

Methods and systems are disclosed for treating injured and/or ischemic tissue by delivering a platelet composition which induces neovascularization in the tissue and improves tissue perfusion.

### CROSS-REFERENCE TO RELATED APPLICATIONS

The present application claims priority under 35 U.S.C. 119(e) to U.S. provisional patent application No. 60/956,754 filed Aug. 20, 2007. The contents of that application are incorporated by reference herein in their entirety.

### FIELD OF THE INVENTION

The present disclosure relates generally to systems and methods for inducing neovascularization in tissues. Specifically, the present disclosure relates to treating peripheral vascular disease. More specifically, the present invention discloses compositions and methods for improving perfusion of tissues.

### BACKGROUND OF THE INVENTION

Peripheral vascular disease (PVD) and related disorders are defined as diseases of blood vessels outside of the heart and central nervous system often encountered as narrowing of the vessels of the limbs. There are two main types of these disorders, functional disease which doesn't involve defects in the blood vessels but rather arises from stimuli such as cold, stress, or smoking, and organic disease which arises from structural defects in the vasculature such as atherosclerotic lesions, local inflammation, or traumatic injury. This can lead to occlusion of the vessel, aberrant blood flow, and ultimately to tissue ischemia.

One of the more clinically significant forms of PVD is peripheral artery disease (PAD) which has elements in common with Coronary Artery Disease (CAD). Similar to CAD, PAD is often treated by angioplasty and implantation of a stent or by artery by-pass surgery. Clinical presentation depends on the location of the occluded vessel. For example, narrowing of the artery that supplies blood to the intestine (e.g., the superior mesenteric artery) can result in severe postprandial pain in the lower abdomen resulting from the inability of the occluded vessel to meet the increased oxygen demand arising from digestive and absorptive processes. Severe forms the ischemia can lead to intestinal necrosis. Similarly, PAD in the leg can lead to intermittent pain, usually in the calf, that comes and goes with activity. This disorder is known as intermittent claudication (IC) and can progress to persistent pain while resting, ischemic ulceration, and even limb-threatening ischemia requiring amputation. Currently available therapeutic interventions for PVD include thrombolytic drugs and anti-thrombotic drugs (heparin, aspirin, coumadin), exercise (for IC), anti-atherogenic drugs (e.g., statins), and surgical revascularization. However, many patients have a form of disease that is not anatomically suitable for surgical intervention.

Peripheral vascular disease is also manifested in atherosclerotic stenosis of the renal artery, which can lead to renal ischemia and kidney dysfunction. Biologic revascularization provides a potential alternative to surgical approaches. It involves the processes of angiogenesis and arteriogenesis which combine to drive development of new collateral blood flow for by-passing blood flow around the occlusion. Biologic revascularization can be achieved by drug and gene therapy providing angiogenic factors or by cellular therapy delivering cells that contribute to angiogenesis by paracrine release of angiogenic factors and/or by providing a source of cells that can form endothelium.

One mode of delivering medical agents to tissue is by direct injection into tissue. Another approach is an intravascular approach. Catheters may be advanced through the vasculature and into the ischemic or injured tissue to inject materials directly into tissue at a treatment site. Furthermore, additional therapies being developed for treating injured and/or ischemic tissue include the injection of cells and/or other biologic agents into ischemic tissue or placement of cells and/or agents onto the ischemic tissue. One therapy for treating ischemic or injured tissue includes the delivery of cells that are capable of maturing into actively contracting muscle cells. Examples of such cells include myocytes, myoblasts, mesenchymal stem cells, and pluripotent cells. Delivery of such cells into tissue is believed to be beneficial.

It has been postulated that after acute or chronic injury, or as a response to disease, endogenous regenerative cells attempt to restore some or all function to the injured tissue. It is likely that the reduced blood flow and vascular supply to the injured region inhibits these recuperative mechanisms. The provision of more adequate perfusion may facilitate earlier, faster, and/or more complete recovery.

A focus therefore remains on re-establishing blood flow to the ischemic zone. Re-establishing blood flow may be accomplished through stimulation of angiogenesis in which the body generates or expands blood supply to a particular region. Prior methods for re-establishing blood flow and rehabilitating the peripheral vasculature frequently involved invasive surgery. Other methods have used lasers to bore holes through the ischemic zones to promote blood flow. These surgeries are complicated and dangerous. Therefore, a need exists for a safe less-invasive method for re-establishing blood flow.

None of these discussed methods specifically induce angiogenesis or neovascularization of the injured tissue. Establishment of an improved or normal blood supply within the injured tissue can prevent further deterioration and promote regeneration of the injured tissue. Such a treatment would be advantageous over previously used treatments. For these reasons, it is desirable to have an agent that could be delivered directly to tissue to induce neovascularization.

### SUMMARY OF THE INVENTION

The present disclosure provides biocompatible compositions for inducing neovascularization in ischemic tissues. Additionally, methods of improving perfusion in tissues are provided. Associated methods and systems for treating patients having tissue injuries and/or peripheral vascular disease are also provided.

In one embodiment, a system for inducing neovascularization in a tissue is provided comprising a platelet composition and at least one delivery device for introducing the platelet composition into the tissue; wherein the platelet composition induces neovascularization in the tissue. In another embodiment, the platelet composition is selected from the group consisting of platelet gel, platelet rich plasma and platelet poor plasma. In another embodiment, the platelet composition is autologous.

In another embodiment, the platelet gel is formed from platelet poor plasma or platelet rich plasma and an activating agent. In another embodiment, the activating agent is thrombin. In another embodiment, the thrombin is selected from the group consisting of recombinant thrombin, human thrombin, animal thrombin, engineered thrombin and autologous thrombin.

In an embodiment, the platelet gel is formed from platelet rich plasma or platelet poor plasma and thrombin at a ratio of between about 5:1 and about 25:1. In another embodiment, the ratio of platelet rich plasma or platelet poor plasma to thrombin is about 10:1.

In another embodiment, the platelet composition comprises platelet rich plasma without an exogenous source of thrombin. In another embodiment, the platelet composition is delivered to said treatment site and forms a gel within said treatment site.

In yet another embodiment, the platelet composition further comprises a bioactive agent selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, cells, and combinations thereof. In another embodiment, the platelet composition further comprises a contrast agent.

In another embodiment, the tissue is in a condition selected from the group consisting of a healthy state, an injured state and an ischemic state at the time of introduction.

In another embodiment, the platelet composition is provided in about 1 to 20 injections. In another embodiment, the injections are provided sequentially. In another embodiment, the injections are provided approximately simultaneously. In another embodiment, the platelet composition comprises a total injection volume up to about 500 mL. In another embodiment, each injection of platelet composition comprises an injection volume up to about 100 milliliters per injection.

In additional embodiments, the delivery device is an injection catheter which introduces platelet composition to the treatment site through an approach selected from trans-arterial approach, trans-venous and trans-cutaneous. In another embodiment, the introduction of said platelet composition to the treatment site is performed with in situ visualization such as echography and ultrasound. In another embodiment, the delivery device comprises lumen in a biaxial or coaxial configuration. In another embodiment, the delivery device comprises staggered or flush tips.

In another embodiment, the platelet composition is introduced to the treatment site at multiple injection sites along a path. In another embodiment, the injection sites are continuous or interrupted along a path. In another embodiment, the platelet composition is administered during needle insertion or during needle pull-back.

In another embodiment, the platelet composition is provided to the treatment site in a patient before the onset of ischemia. In another embodiment, the platelet composition is provided to the treatment site after the onset of limb-threatening ischemia. In one embodiment, the patient is at risk for peripheral vascular disease and the platelet composition is provided to the treatment site before the onset of ischemia.

In another embodiment, the treatment site is selected from the group consisting of the ischemic area, the peri-ischemic area and the healthy tissue surrounding the ischemic area.

In one embodiment, a method of inducing neovascularization in tissue is provided comprising providing a platelet composition at a treatment site in the tissue wherein the platelet composition induces neovascularization in the tissue. In another embodiment, the platelet composition improves tissue perfusion. In another embodiment, the platelet composition is selected from the group consisting of platelet gel, platelet rich plasma and platelet poor plasma. In another embodiment, the platelet composition is autologous.

In one embodiment, a method of treating peripheral vascular disease is provided comprising providing a platelet composition into a treatment site in ischemic tissue wherein the composition induces neovascularization of the tissue; and injecting a cell preparation into the re-vascularized tissue. In another embodiment, the platelet composition is selected from the group consisting of platelet gel, platelet rich plasma and platelet poor plasma. In another embodiment, the platelet composition is autologous. In another embodiment, the platelet gel is formed from platelet poor plasma or platelet rich plasma and an activating agent. In another embodiment, the activating agent is thrombin. In another embodiment, the thrombin is isolated from a source selected from the group consisting of recombinant thrombin, autologous thrombin, bovine thrombin, human thrombin, mammalian thrombin, and engineered thrombin.

In another embodiment, the platelet composition comprises platelet rich plasma without an exogenous source of thrombin.

In another embodiment, the platelet composition further comprises a bioactive agent selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof. In another embodiment, the cell preparation comprises cells of one or more cell types selected from the group consisting of somatic, germ-line, fetal, embryonic, post-natal cells and adult cells. In another embodiment, the cell preparation comprises cells isolated from one or more tissue types selected from the group consisting of adipose, brain, muscle, endothelial, blood, bone marrow, heart, testes and ovaries. In another embodiment, the cells are autologous. In another embodiment, the cells are modified prior to implantation. In another embodiment, the cell preparation further comprises a bioactive agent. In another embodiment, the cell preparation further comprises a platelet composition. In another embodiment, the cell preparation is provided to the tissue after neovascularization is initiated in said tissue. In another embodiment, the platelet composition and said cell preparation are provided to the tissue approximately simultaneously.

In another embodiment, the treatment site is selected from the group consisting of the ischemic area, the peri-ischemic area and the healthy tissue surrounding the ischemic area. In another embodiment, the platelet composition and said cell preparation are injected into the same treatment site. In another embodiment, the platelet composition and said cell preparation are injected into different treatment sites. In another embodiment, the cell preparation is injected adjacent to the site of injection of said platelet gel composition.

### BRIEF DESCRIPTION OF THE DRAWINGS

FIG. 1 depicts an occluded artery in the leg of a patient with peripheral vascular disease.

FIG. 2 is a block diagram showing the steps of treating ischemic or injured tissue.

FIG. 3 schematically depicts a device for delivery of a composition into tissue.

FIG. 4 schematically depicts a detailed view of delivery of a composition into tissue.

FIG. 5 schematically depicts the migration of a composition within the muscle tissue after delivery.

FIG. 6 depicts an embodiment of the present invention wherein a composition is delivered in multiple doses along tracks leading to the parent artery.

FIG. 7 depicts the transcutaneous administration of a composition to a target tissue.

FIG. 8 depicts the transvenous administration of a composition into a target tissue.

FIG. 9 depicts the transarterial administration of a composition into a target tissue.

FIG. 10 depicts a photomicrograph of infarcted myocardium eight weeks after injection with autologous platelet gel (platelet rich plasma and bovine thrombin at 10:1 ratio) delivered one hour after infarction. Many blood vessels (arrow A) are observed within a region of infarcted tissue (arrow C). These vessels are carrying red blood cells (arrow B).

FIG. 11 depicts a higher magnification photomicrograph of infarcted myocardium eight weeks after injection with autologous platelet gel (platelet rich plasma and bovine thrombin at 10:1 ratio) delivered one hour after infarction. Many blood vessels (arrow A) are observed within a region of infarcted tissue (arrow C). These vessels are carrying red blood cells (arrow B).

### DEFINITION OF TERMS

Generally, all technical terms or phrases appearing herein are used as one skilled in the art would understand to be their ordinary meaning. For the convenience of the reader, however, selected terms are more specifically defined as follows.

Angiogenesis: As used herein, “angiogenesis” refers to a physiologic process involving the growth of new blood vessels from pre-existing blood vessels.

Bioactive agent: As used herein, “bioactive agent” includes therapeutic agents and drugs and includes pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, inhibitors of compounds implicated in remodeling (e.g., inhibitors of angiotensin II, angiotensin converting enzyme, atrial natriuretic peptide, aldosterone, renin, norepinephrine, epinephrine, endothelin, etc.) and combinations thereof.

Composition: As used herein, “composition” refers to an injectate, substance or a combination of substances which can be delivered into a tissue and are used interchangeably herein. Exemplary compositions include, but are not limited to, platelet gel, autologous platelet gel, platelet rich plasma and platelet poor plasma, with and without the addition of bioactive agents, structural materials, etc.

Delivery: As used herein, “delivery” refers to providing a composition to a treatment site in an injured tissue through any method appropriate to deliver the functional composition to the treatment site. Non-limiting examples of delivery methods include direct injection at the treatment site, direct topical application at the treatment site, percutaneous delivery for injection, percutaneous delivery for topical application, and other delivery methods well known to persons of ordinary skill in the art.

Improves: As used herein when referring to tissue perfusion, the term “improves”, defines a level of perfusion that is increased when compared to the same tissue before treatment.

Injury area: As used herein, “injury area” refers to the injured tissue. The “peri-injury area” refers to the tissue immediately adjacent to the injured tissue, that is, the tissue at the junction between the injured tissue and the normal tissue.

Injured tissue: As used herein, “injured tissue” refers to tissue injured by trauma, or disease and includes ischemic tissue, infarcted tissue or tissue damaged by any means which results in interruption of normal blood flow to the tissue. This includes tissue with insufficient arterial blood supply or inadequate venous drainage or both.

Ischemia: As used herein. “ischemia” or “ischemic tissue” refers to tissue having a relative shortage of the blood supply and therefore inadequate oxygen supply. Ischemia can also be described as an inadequate flow of blood to a part of the body, caused by constriction or blockage of the blood vessels supplying it. Ischemia results in the death of tissues.

Neovascularization: As used herein, “neovascularization” refers to the formation of functional vascular networks that may be perfused by blood or blood components. Neovascularization includes angiogenesis, budding angiogenesis, intussuceptive angiogenesis, sprouting angiogenesis, therapeutic angiogenesis, arteriogenesis, and vasculogenesis.

Peripheral vascular disease: As used herein, the term “peripheral vascular disease” refers to a variety of diseases caused by the obstruction of large peripheral arteries, which can result from atherosclerosis, inflammatory processes leading to stenosis, an embolism or thrombus formation. Peripheral vascular disease causes acute or chronic ischemia. Peripheral vascular disease also includes peripheral artery disease.

Percutaneous: As used herein, the term “percutaneous” refers to any penetration through the skin of the patient, whether in the form of a small cut, incision, hole, cannula, tubular access sleeve or port or the like. A percutaneous penetration may be made in an interstitial space between the ribs of the patient or it may be made elsewhere, such as the groin area of a patient.

Restores: As used herein when referring to tissue perfusion, the term “restores” defines a level of perfusion that is increased when compared to the same tissue before treatment when the tissue pre-treatment is ischemic and the tissue post-treatment has increased perfusion not exceeding that of normal tissue.

Tissue injury: As used herein, “tissue injury” refers to any area of abnormal tissue caused by a disease, disorder or injury and includes damage to the epithelia and/or muscle. Non-limiting examples of causes of tissue injury include acute or chronic stress (systemic hypertension, diabetes), peripheral vascular disease, ischemia or infarction, and inflammatory diseases. Furthermore, there are occasions when the injury is acute, where the injury may be referred to as an injurious event. Injured tissue includes tissue that is ischemic, infarcted, or otherwise focally or diffusely diseased.

Tissue Perfusion: As used herein, “tissue perfusion” refers to the availability of blood to target tissue.

Vasculogenesis: As used herein, the term “vasculogenesis” refers to blood vessels formation by de novo production of endothelial cells, a process that occurs during development and also in adulthood (e.g. after trauma, injury or disease).

### DETAILED DESCRIPTION

The present disclosure provides biocompatible compositions for inducing neovascularization to improve tissue perfusion in ischemic or normal tissues. Associated methods and systems for treating patients with tissue injuries and/or peripheral vascular disease are also provided.

After an injury, such as but not limited to an ischemic insult, atherosclerosis or disease, blood supply to the tissue is often insufficient to meet tissue demands under rest and/or high-demand conditions. Persistently ischemic tissue can die and neighboring tissue is at increased risk of ischemia. This process may result in the growth of the area of ischemia with time. Increasing the blood supply to the damaged or neighboring tissue can prevent further ischemia.

In embodiments of the present disclosure, compositions and methods are provided for inducing angiogenesis in injured and/or ischemic tissue by injecting a platelet composition directly into the injured or surrounding tissue to produce a collateral network of arteries, arterioles and capillaries which are connected to a parent artery. In one embodiment, the platelet composition induces neovascularization. In another embodiment, the platelet composition induces neovascularization to regenerate injured tissue. In yet another embodiment, the platelet composition induces neovascularization to promote regeneration of tissue or function.

Additionally, the compositions and methods can be used to enhance perfusion in normal tissue. Induction of angiogenesis can lead to increased perfusion of tissues and therefore improved function, increased stamina and improved performance by the tissues.

Neovascularization refers to the development of new blood vessels from endothelial precursor cells by any means, such as by vasculogenesis, angiogenesis, or the formation of new blood vessels from endothelial precursor cells that link to existing blood vessels. Angiogenesis is the process by which new blood vessels grow from the endothelium of existing blood vessels in a developed animal. Endothelial precursor cells circulate in the blood and selectively migrate, or “home,” to sites of active neovascularization (see U.S. Pat. No. 5,980,887, Isner et al., the contents of which are incorporated herein by reference in their entirety).

The disclosed methods and system for inducing neovascularization can also be used to treat other conditions besides peripheral vascular diseases. In one embodiment, the disclosed methods and systems are useful to induce neovascularization in gastrointestinal organs. Non-limiting examples of gastrointestinal use of the instant systems include injecting or applying the platelet composition to the stomach or intestine wall and injecting the platelet composition into the liver.

In another embodiment, the systems and methods are useful to treat or heal wounds in the skin. Skin wounds can exhibit delayed or impaired healing due to an underlying ischemia. In non-limiting examples, the platelet compositions are applied or injected into, around or beneath skin wounds to promote neovascularization and healing.

In another embodiment, the disclosed system and methods are useful for inducing neovascularization in any muscle tissue that is ischemic. Examples of muscle tissue which can be treated with the instant methods and system includes, but is not limited to, skeletal muscle such as muscles in the limbs and the tongue, cardiac muscle and smooth muscle.

The present methods and compositions will now be described in detail below by reference to the drawings.

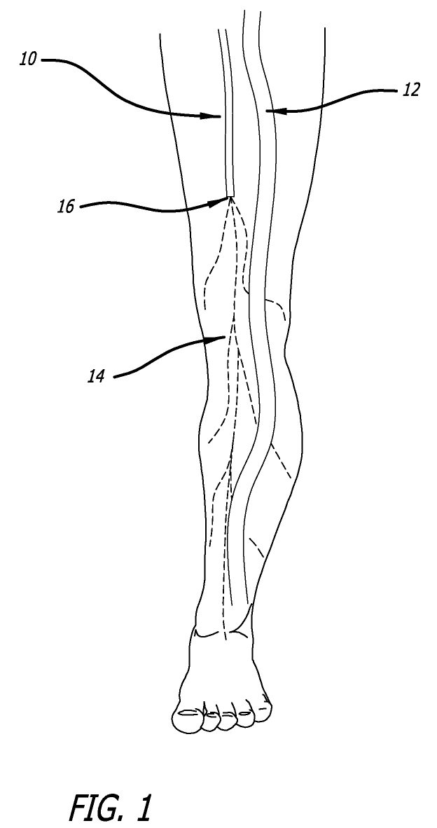

FIG. 1 graphically depicts the leg of a subject having an artery 10 and a vein 12. Artery 10 branches in a series of arterial branches 14. In FIG. 1, artery 10 is blocked at site 16, preventing flow of blood past site 16 and through obliterated arterial branches 14. Tissue fed by artery 10 and arterial branches 14 becomes ischemic as a result of the reduced blood flow.

In the absence of adequate blood flow in the injured region, endogenous repair mechanisms are not able to restore tissue function. Endogenous cells have been demonstrated to “home” to injured tissue, even in the adult, but blood flow limitations may prevent them from taking residence and promoting healing.

As described further below, embodiments disclosed herein address peripheral vascular disease by injecting a composition into the injured and/or ischemic tissue to induce neovascularization and thus restore function. Also contemplated is providing neovascularization to any ischemic and/or injured tissue in need of revascularization.

For the purpose of this document, the term “platelet gel” refers to platelet compositions which are administered with an activating agent and may provide biological therapy such as neovascularization. Furthermore, the platelet composition can refer to platelet rich or platelet poor plasma that is administered without an activating agent. Platelet compositions such as platelet rich (PRP) and platelet poor plasma (PPP) can additionally be activated by tissue thrombin in situ to provide neovascularization. Exemplary, non-limiting platelet compositions include platelet gel, autologous platelet gel (APG), platelet rich plasma, and platelet poor plasma.

Before any composition is injected into a region of injured tissue, to induce neovascularization of the tissue, the location and extent of the injured region may be identified. Multiple technologies and approaches are available for the clinician to identify and assess normal versus ischemic tissue. These include, but are not limited to, visual inspection, localized blood flow determinations, local electrical and structural activity, electromyography (EMG), nuclear imaging, angiography, ultrasound imaging, magnetic resonance imaging (MRI), positron emission tomography (PET) scans, and computerized tomography (CT) scans.

In one embodiment, the platelet compositions are prepared by using the Medtronic Magellan® Platelet Separator. Anticoagulated whole blood is prepared by combining an anticoagulant with whole blood freshly removed from the subject. The Magellan® device is used to then extract platelet rich plasma (PRP) and platelet poor plasma (PPP) from the sample of anticoagulated whole blood. Platelet gel is prepared by combining the resulting PRP or PPP with an activator. In one embodiment the activator is bovine thrombin which has been reconstituted to 1000 Units/milliliter in 10% calcium chloride solution. In another embodiment, PRP is combined in an approximately 10:1 ratio with bovine thrombin. In yet another embodiment, the activator is human thrombin, such as, but not limited to, autologous thrombin.

In one embodiment, the composition is a platelet gel that is made using a PRP to thrombin ratio of about 10:1. Another embodiment uses a PRP to thrombin ratio of about 11:1. Other embodiments of the present invention have ratios of PRP to thrombin of about 5:1 to about 25:1. In another embodiment, the ratio of PRP to thrombin is about 7:1 to about 20:1. In another embodiment, the ratio of PRP to thrombin is about 9:1 to about 15:1. In another embodiment, the ration of PRP to thrombin is about 10:1 to about 12:1. In at least one embodiment, no thrombin is included and PRP is injected into the tissue alone. Other embodiments include multiple components of the composition in ratios needed to achieve or optimize the desired effect.

The PRP contains a high concentration of platelets that can aggregate during gelling, as well as release cytokines, growth factors or enzymes following activation. Some of the many factors released by the platelets and the white blood cells that constitute the PRP include platelet-derived growth factor (PDGF), platelet-derived epidermal growth factor (PDEGF), fibroblast growth factor (FGF), transforming growth factor-beta (TGF-β) and platelet-derived angiogenesis growth factor (PDAF). These factors have been implicated in wound healing by increasing the rate of collagen secretion, vascular in-growth and fibroblast proliferation.

Once the location, size and shape of the target region are identified, the clinician can access and begin injecting the tissue with the platelet compositions. In one embodiment, the platelet composition comprises PRP and thrombin. In another embodiment, the platelet composition comprises PRP alone. In another embodiment, the platelet composition comprises PPP and thrombin. In yet another embodiment, the platelet composition comprises PPP alone. The components of the platelet composition may be derived from humans, and/or animals, and/or recombinant sources. The components may also be artificially produced or fortified. The components for platelet composition can be categorized as autologous, or non-autologous, and the non-autologous components can be further categorized as described above (i.e., animal, recombinant, engineered, allogeneic human, etc.). Autologous platelet gel (APG) refers to a composition made from autologous PRP or autologous PPP and an autologous or non-autologous activator. One advantage in using autologous and/or recombinant components in the injected compositions is that it reduces the recipient's risk of an inflammatory response or exposure to infectious and foreign agents.

Additionally, the platelet composition can include one or more bioactive agents to induce healing or regeneration of damaged tissue. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. The platelet composition may also include cellular additives such as stem cells, leukocytes, red blood cells, neuronal precursor cells, muscle cell precursors, cultured cells, or other differentiated or undifferentiated cells.

Furthermore, the platelet compositions can include a contrast agent for detection by X-rays, magnetic resonance imaging (MRI) or ultrasound. Suitable contrast agents are known to persons of ordinary skill in the art and include, but are not limited to, radiopaque agents, echogenic agents and paramagnetic agents. A contrast agent may be used in the composition of some embodiments for visual confirmation of injection success. Examples of such contrast agents include, but are not limited to, X-ray contrast (e.g., IsoVue or other contrast agents having a high X-ray attenuation coefficient), MRI contrast (e.g., gadolinium or other contrast agents detectable as signal or signal-void by MRI), and ultrasound contrast (echogenic or echo-opaque compounds).

When the PRP and thrombin are injected such that they mix in the tissue (see description of delivery devices below) they will gel in the tissue. Several embodiments of the present invention provide accelerated gel times. The gelling time in situ can be accelerated by applying local heat to the injection site via a delivery catheter or other instrument, increasing the thrombin concentration, or combining the PRP and thrombin in a mixing chamber and injecting the mixture into the tissue after the mixture has begun gelling. This description also applies for other multi-component compositions, where the components gel, cross-link and/or polymerize after being mixed together.

As described in further detail in the Examples, the platelet compositions of the present invention have been injected into normal and ischemic cardiac muscle tissue of test subjects (sheep and pigs). The experiments indicate that injections of PRP and thrombin are safe and well tolerated when made into infarcted or non-infarcted tissue, and that they can be performed safely as early as 1 hr after infarction (MI). Injections were made both orthogonally and obliquely to the heart muscle surface at intervals of 0.5 to 2.5 cm. A plurality of injections can be made without safety problems. Within the small target area of the heart, the total injectate volume was tested to be safe as high as 15.0 mL, and the volume of individual injections were tested to be safe as high as 1100 μl per injection site. It is anticipated that in peripheral tissues, much larger doses could be safely tolerated. Since peripheral tissues are also substantially thicker than myocardial wall, injectate could be delivered at multiple sites along a needle's path, for example by interrupted or continuous injections administered during needle insertion or needle pull-back.

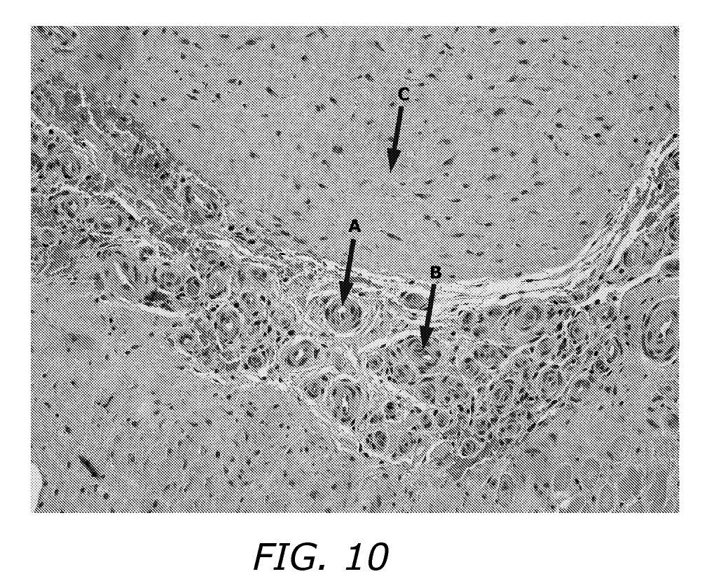

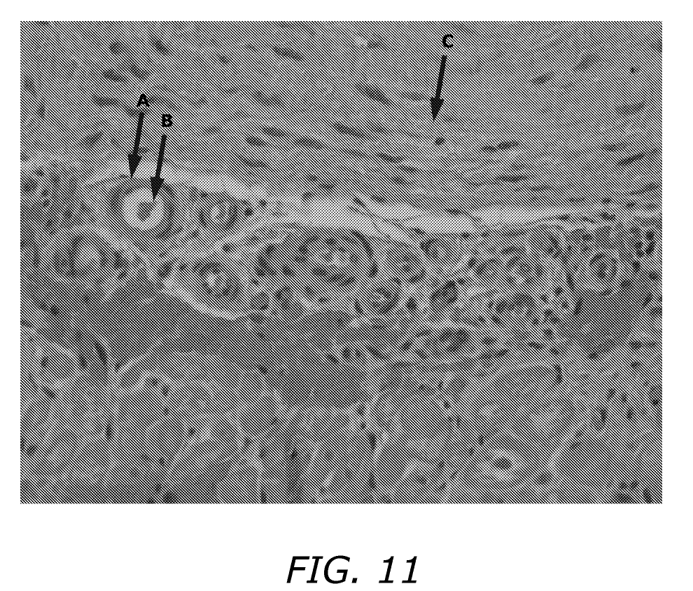

Furthermore, APG administration following cardiac injury partially or fully reversed detrimental acute effects of infarction on the ejection fraction (EF) and augmented EF towards or above pre-infarct levels. APG administration following myocardial injury into ischemic tissue stimulated neovascularization of the injured tissue (FIGS. 10-11). The extent of vascularization was markedly and statistically significantly greater than in infarcted animals not receiving platelet gel therapy. All or a subset of the components of platelet gel (PRP or PPP components with or without thrombin) may be used to generate such an effect.

In order to practice the presently disclosed methods and deliver a platelet composition to sites within a target tissue, a clinician may use one of a variety of access techniques.

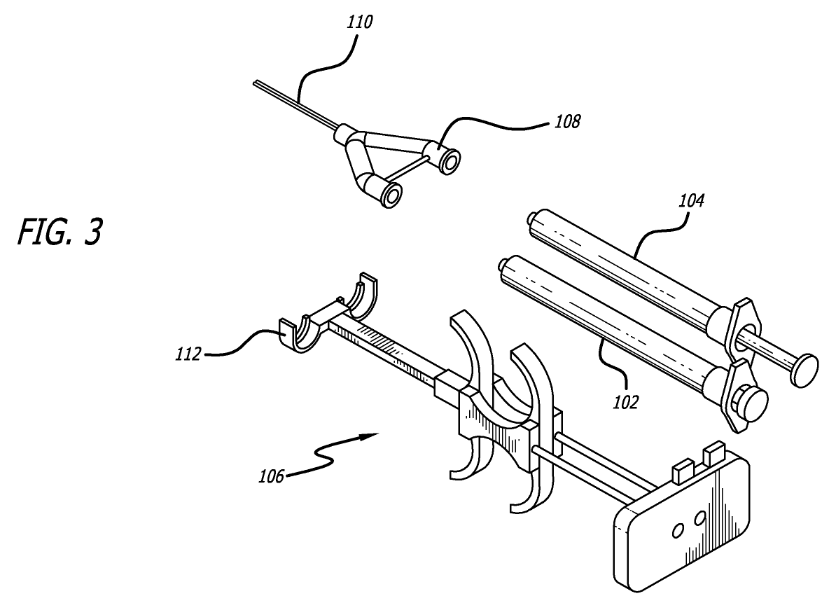

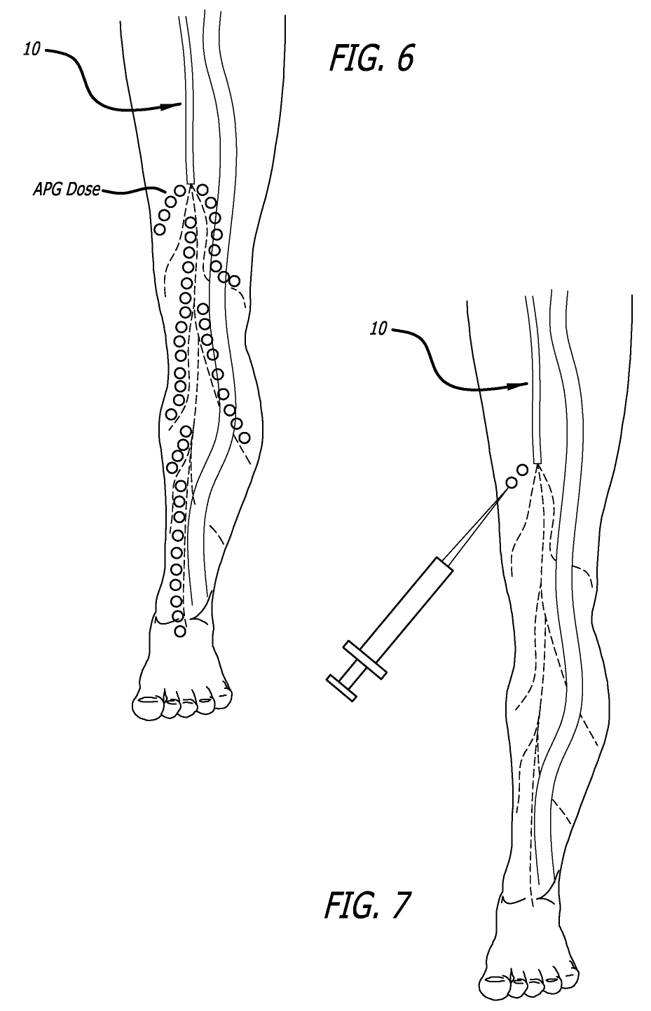

FIG. 6 depicts one embodiment wherein a composition, such as but not limited to APG, is injected in multiple doses in ischemic tissue along tracks leading to parent artery 10 to encourage APG-induced arteries to grow between the target ischemic tissue and the parent artery. Delivery methods for administration of the APG to the ischemic tissue are depicted in FIGS. 3-5.

FIG. 7 depicts the transcutaneous administration of a composition by injecting the composition through the skin into the ischemic tissue along a path leading to the parent artery. The injectate may be delivered at multiple sites along a needle's path, for example by interrupted or continuous injections administered during needle insertion or needle pull-back. The transcutaneous injection(s) can be optionally guided by ultrasound or fluoroscopy. An echogenic or radio-opaque needle may be used.

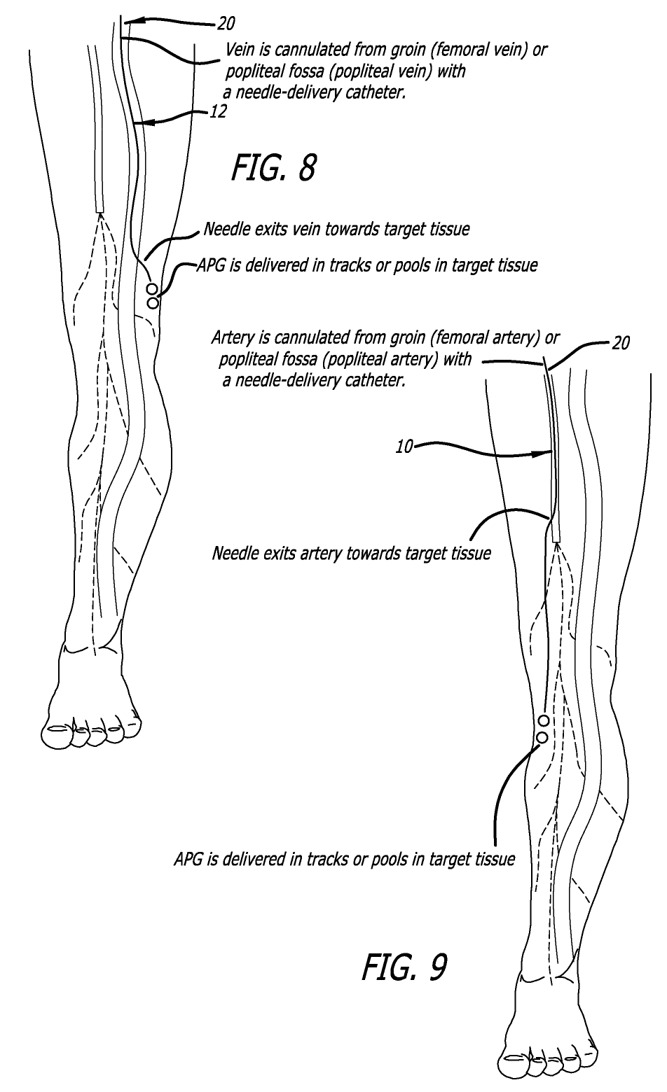

FIG. 8 depicts the transvenous administration of a composition by cannulating vein 12 from the groin (femoral vein) or popliteal fossa (popliteal vein) with an injection catheter 20. The needle of injection catheter 20 penetrates vein 12 adjacent to target tissue and administers the composition in tracks or pools in the target tissue. The transvenous injection(s) can be optionally guided by ultrasound and or fluoroscopic guidance means. The ultrasound capability may be present on the device (an intravascular ultrasound system) or externally.

FIG. 9 depicts the transarterial administration of a composition by cannulating artery 10 from the groin (femoral artery) or popliteal fossa (popliteal artery) with an injection catheter 20. The needle of injection catheter 20 penetrates artery 10 adjacent to target tissue and administers the composition in tracks or pools in the target tissue. The transarterial injection(s) can be optionally guided by ultrasound and or fluoroscopic guidance means. The ultrasound capability may be present on the device (an intravascular ultrasound system) or externally.

At least one embodiment includes two or more side-by-side syringes for one-handed injection of the multiple composition components. In one embodiment, the device of FIG. 3 is used to inject a multi-component composition into injured and/or ischemic tissue. In the embodiment of FIG. 3, two components of the composition of the present invention are housed separately in syringes 102 and 104. Syringes 102 and 104 are disposed in cradle 112 within a handle assembly 106 to allow one-handed injection of the composition. An adapter 108 couples to the syringes 102 and 104 to a biaxial needle 110. Biaxial needle 110 allows the delivery of two components of a composition, in a non-limiting example, PRP and thrombin, to a treatment site.

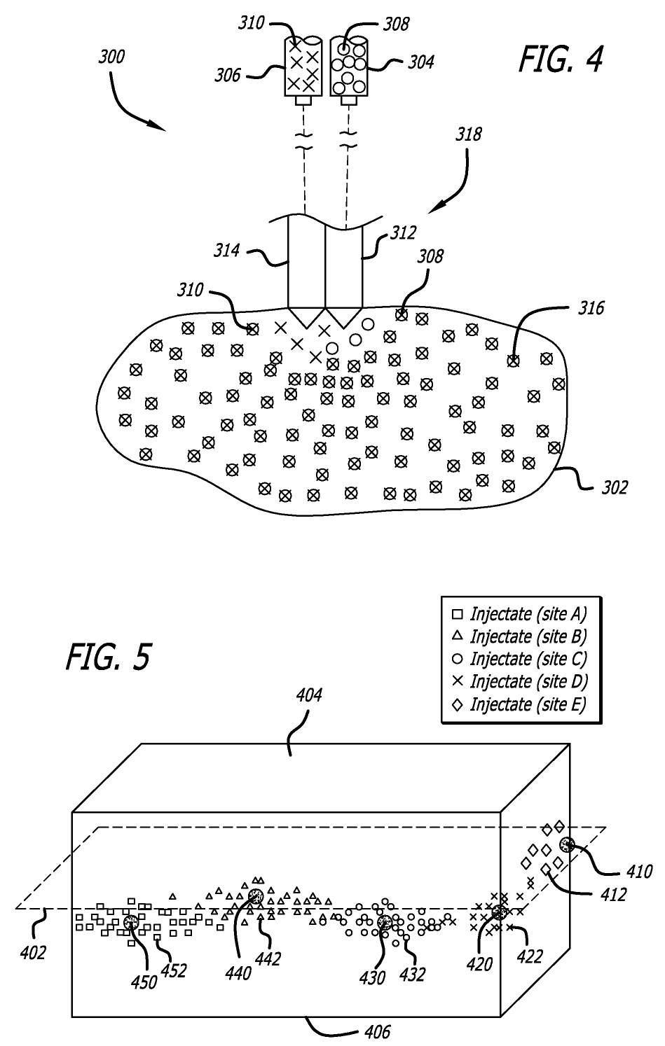

FIG. 4 represents an enlarged view of the injection of a two-component composition using a biaxial injection needle containing delivery device 300. Component 310 is held in reservoir or syringe 306 and component 308 is held in reservoir or syringe 304. Components 310 and 308 are caused to pass into biaxial needle 318 comprising needle lumen 314 for injection of component 310 and needle lumen 312 for injection of component 308. Components 310 and 308 are injected into the treatment site 302 simultaneously and the two components combine to form composition 316. Immediately after injection, components 310 and 308, and to a certain extent composition 316 diffuse through the tissue at treatment site 302. The components and compositions have been observed to diffuse up to two centimeters in tissue. In another embodiment, the needle 318 is co-axial and has two lumens.

The delivery system may deliver the components of the composition in a prescribed ratio. This ratio may be pre-set (and fixed) or dialable (and dynamic). One embodiment utilizes separate gears or levers (with gear-ratio or lever-ratio that are settable) to enable delivery of multiple compounds in different ratios without generating a pressure gradient between syringes. Other multi-component delivery devices include lumens of different caliber to allow for pre-determined ratio of each component. Some multi-component delivery devices include lumens of different lengths, such that one component is released more distally than another. Still other devices incorporate one or more mixing chambers in the device. At least one embodiment of the delivery devices includes single lumen needle/catheters that are used for serial delivery of multiple components (one after another).

Several embodiments of delivery devices can be placed in a vessel neighboring the target treatment site and used to deliver platelet compositions to the tissue by piercing through the vessel wall and navigating to the desired location with the needle-tip or a microcatheter that is contained in the needle. The catheter or needle may contain a local imaging system for identifying the target area and proper positioning of the delivery device. The device may include one or more needles having a closed distal tip and one or more side openings for directing a substance substantially laterally from the distal tip into the tissue. Preferably, the needle has a sufficiently small gauge diameter such that the needle track in the tissue is substantially self-sealing to prevent escape of the composition upon removal of the needle. Recent data (obtained in the context of epicardial delivery) demonstrated hemostasis in vivo when platelet gel was injected through even a large 18 gauge injection needle. This result could be attributable to the rapid coagulation achieved by the components injected and the inherent hemostatic properties of platelet gel. In another embodiment, the needle gauge is smaller than 18 gauge. In one embodiment, the needle is 21 gauge. In another embodiment, the needle gauge is 26 gauge. In another embodiment, the needle is biaxial. In another embodiment, the needle is coaxial.

Alternatively, the delivery assembly may include one or more needles having a plurality of lumens that extend between a multiple line manifold on the proximal end to adjacent outlet ports. A multi-lumen needle assembly may allow components of a substance to be independently injected, thereby allowing the components to react with one another following delivery within the selected tissue region, as described herein.

In one embodiment, a multi-lumen needle assembly may allow two components of a composition to be simultaneously, independently injected, which may then react with one another once within the selected tissue region, as described herein. In another embodiment having a multi-lumen needle assembly, the lumens empty into a mixing chamber located near the distal tip of the needle and the components of the injected substance are mixed with each other immediately prior to being injected into the selected tissue region.

Platelet compositions can be delivered to the target tissue by a catheter system. Suitable catheter delivery systems include systems having multiple biaxial or coaxial lumens with staggered or flush tips. The catheter systems can include needles or other injection devices located at the distal end, and syringes at the proximal end of the catheters. The catheters and other delivery devices can have differently sized lumens to ensure that multi-component compositions can be delivered to the tissue in the desired ratio. Another embodiment of a catheter system may be used to create a composition reservoir within the tissue itself to provide sustained delivery. A catheter may be introduced endovascularly into a blood vessel until the distal portion is adjacent the desired treatment location. The needle assembly may be oriented and deployed to puncture the wall of the vessel and enter the tissue. The composition can then be injected into the tissue and, thereby, form a reservoir.

Devices for injecting the platelet compositions can include refrigerated parts for keeping the various components of the compositions cool. Various embodiments of delivery devices for practicing the current methods can include a refrigerated/cooled chamber for thrombin refill, a refrigerated/cooled chamber for thrombin, and/or an agitator mechanism in a PRP refill or injection chamber to prevent settling of the PRP. Delivery devices can include heating or cooling devices used to heat or cool the tissue or compositions to speed up or slow down the gelling/hardening time after delivery. Some devices can include catheters or other delivery devices with a cooled lumen or lumens for keeping components of the injected compositions cool while they are traveling through a device lumen. As noted above, some devices can include a mixing chamber for mixing the components of an injected composition before the substance is delivered into the tissue. In one embodiment, the PRP is stored in an agitating/vibrating chamber that provides sufficient agitation to keep the PRP homogeneous. In another embodiment, the clinician provides sufficient agitation to the delivery device by tilting, or otherwise manipulating the device to keep the PRP homogeneous.

A clinician practicing the currently disclosed methods may need to make multiple injections using a single delivery assembly. Thus, at least one embodiment of the delivery devices includes a device having at least one reusable needle. Some embodiments may include delivery devices having an automated dosing system, e.g., a syringe advancing system. The automated dosing system may allow each dose to be pre-determined and dialed in (can be variable or fixed), e.g., a screw-type setting system. One embodiment may include a proximal handle wherein each time the proximal handle is pushed; a pre-determined dose is delivered at a pre-determined or manually-controllable rate.

In further alternative embodiments, the delivery system may include a plurality of needle assemblies (similar to the individual needle assemblies described above), to be deployed in a predetermined arrangement along the periphery of a catheter. In one embodiment, the needle assemblies may be arranged in one or more rows. In particular, it may be desirable to access an extended remote tissue region, for example extending substantially parallel to a vessel, within the tissue. With a multiple needle transvascular catheter system, a single device may be delivered into a vessel and oriented. The array of needles may be sequentially or simultaneously deployed to inject a composition into the extended tissue region, thereby providing a selected trajectory pattern. Catheter based devices such as those described above are disclosed in U.S. Pat. No. 6,283,951, the disclosure of which is incorporated herein by reference thereto.

If a clinician is practicing the current methods using a minimally invasive or percutaneous technique, he/she may need some sort of real-time visualization or navigation to ensure site-specific injections. Thus, at least one embodiment uses Medtronic Navigation technologies to superimpose pre-operative Ultrasound, CT, or MRI images onto fluoroscopic images of a delivery catheter to track it in real-time to target sites. In one embodiment, the clinician uses a contrast agent and/or navigation technologies to track the needle-tip during injection in a virtual 3-D environment. This technique marks previous injections to ensure proper spacing of future injections. Completed injections may be tracked using a contrast material injected along with the therapeutic injectate. This contrast material may be an ultrasound, fluoroscopic, x-ray, CT, MRI or other contrast agent.

The needle assembly (or other device component) may include a feedback element or sensor for measuring a physiological condition to guide delivery of compositions to the desired location. During treatment, for example, the composition may be delivered into a tissue region until a desired condition is met.

Regardless of the device used to deliver the platelet composition or how the clinician accesses the target tissue, a clinician practicing the current methods may have the need for precise local placement of each injection. In one embodiment, the substance is delivered/injected to a location in the tissue that is very near to an existing blood supply. In other embodiments, the substances are delivered to a location that is further away from an existing blood supply. In yet another embodiment, the substances are delivered to a location substantially equidistant to more than one existing blood supply.

To achieve depth control during platelet composition administration using the transcutaneous approach, the delivery device of at least one embodiment of the present disclosure includes a stopper fixed (or adjustably fixed) on the needle shaft, at a desired distance from needle's distal tip, to prevent penetration into tissue beyond a specified depth. Some embodiments use the method of injecting one or more needles into tissue at a tangent to the tissue surface to control the depth of the injection. In at least one embodiment of the present invention, the needle can be positioned to inject at an angle perpendicular (90 degrees) to the tissue, tangential (0 degrees) to the tissue, or any desired angle in between. Suction can facilitate controlled positioning and entry of the injector.

At least one embodiment uses a “Smart-Needle” to guide the needle tip to the desired location(s) within the tissue. Such a needle can rely on imaging around or ahead of the needle tip by imaging modes such as ultrasound.

At times it might be desirable to distribute the platelet composition as widely as possible around the injection site. It might also be desirable to have the platelet composition be uniformly distributed around the injection site. One method for enhancing distribution of a platelet composition around an injection site is to use needles having holes in the side vs. using needles having holes in the end. Multiple side holes can provide a wider distribution of composition around the injection site. Side holes also provide access to the tissue from a multitude of places rather than just from the end of the needle, thereby requiring less travel of the composition for wider distribution. Another method for enhancing distribution of a composition around an injection site is to increase the number of needles used at the injection site. If desired, the multi-needle delivery device of the present invention, allows for multiple needles to be placed close to each other in order to provide a uniform distribution over a larger area as compared to the use of a single needle device. The combination of side holes on the needles of a multi-needle device may provide a broad distribution of composition around an injection site.

In one embodiment, suction may be used to improve the distribution of a composition around the injection site. The use of suction can create a negative pressure in the interstitial space. This negative pressure within the interstitial space can help the composition to travel farther and more freely, since the composition is driven by a negative pressure gradient. The combination of suction and side holes on the needles of a multi-needle device may provide a more thorough and broad distribution of composition around an injection site.

In one embodiment, the delivery of platelet compositions from the delivery device into tissue may be enhanced via the application of an electric current, for example via iontophoresis. In general, the delivery of ionized agents into tissue may be enhanced via a small current applied across two electrodes. Positive ions may be introduced into the tissue from the positive pole, or negative ions from the negative pole. The use of iontophoresis may markedly facilitate the transport of certain ionized agents through tissue.

In one embodiment, one or more needles of the delivery device may act as the positive and/or negative poles. For example, a grounding electrode may be used in combination with a needle electrode via a monopolar arrangement to deliver an ionized composition iontophoretically to the target tissue. In one embodiment, a composition may be first dispersed from the needle into tissue. Following delivery, the composition may be iontophoretically driven deeper into the tissue via the application of an electric current. In one embodiment, a delivery device having multiple needles may comprise both the positive and negative poles via a bipolar arrangement. Further, in one embodiment, multiple needle electrodes may be used simultaneously or sequentially to inject a substance and/or deliver an electric current.

When practicing the current methods, one goal is to inject a substance into the target tissue while avoiding accidental delivery into the vascular system. Delivery into one or more of these areas may have negative consequences such as pulmonary or systemic embolization, stroke, and/or distant thromboembolism, for example. The current methods addresses and attempts to prevent these negative consequences in a variety of ways. In at least one embodiment, the ratio of the components of the composition is selected so that the composition gels or polymerizes almost immediately in situ to minimize migration of one or more of the components. One embodiment uses a “Smart Needle” as described above to prevent negative consequences from occurring.

At least one embodiment includes a proximally-hand-operated distal sleeve that covers the needle tip or applies local negative pressure to prevent outward flow of component(s) from the tip of the needle between injections where multiple injections are required. In at least one embodiment, the column of components in a catheter is held under a constant minimum pressure that prevents outflow in-between injections. In at least one embodiment, one-way valves may be placed within each line to prevent entry of one component into a line containing another. This is especially important when the gelling reaction is rapid and the different components need to be maintained separately until the time and site of injection. This will prevent clogging of the delivery device, which will allow repeated injections using a single device.

At least one embodiment prevents backbleed out of the needle track, during and after removal of the needle, by keeping the needle in place for several seconds (e.g. 5-30 sec beyond the expected clotting time) following injection, to utilize the injectate as a ‘plug’ preventing back-bleed, before needle removal. In at least one embodiment, the needle is left in place for the expected gelling time of the injected substance and then withdrawn. In one embodiment, the gelling time of an injected composition is five seconds.

Several embodiments of the current disclosure can include sensors and other means to assist in directing the delivery device to a desired location, ensuring that the injections occur at a desired depth, ensuring the delivery device is at the treatment site, ensuring that the desired volume of composition is delivered, and other functions that may require some type of sensor or imaging means to be used. For example, real-time recording of electrical activity, pH, oxygenation, metabolites such as lactic acid, CO₂, or other local indicators of tissue viability or activity can be used to help guide the injections to the desired location. In some embodiments, the delivery device may include one or more sensors. For example, the sensors may be one or more electrical sensors, fiber optic sensors, chemical sensors, imaging sensors, structural sensors and/or proximity sensors that measure conductance. In one embodiment, the sensors may be tissue depth sensors for determining the depth of tissue adjacent the delivery device. In one embodiment, a sensor that detects pH, oxygenation, a blood metabolite, a tissue metabolite, etc may be used at the end of the delivery device to alert the user if and when the tip has entered the bloodstream (e.g. a vascular structure). This would cause the operator to re-position the delivery instrument before delivering the composition. The one or more depth sensors may be used to control the depth of needle penetration into the tissue. In this way, the needle penetration depth can be controlled, for example, according to the thickness of the target tissue. In some embodiments, sensors may be positioned or located on one or more needles of the delivery device. In some embodiments, sensors may be positioned or located on one or more tissue-contacting surfaces of the delivery device. In other embodiments, the delivery device may include one or more indicators. For example, a variety of indicators, e.g., visual or audible, may be used to indicate to the physician that the desired tissue depth has been achieved. In some embodiments, the sensor or guiding systems may be separate from the delivery system, for example an ultrasound unit.

Furthermore, the delivery device may comprise sensors to allow the surgeon or clinician to ensure the delivery device is within the tissue rather than in a vascular lumen at the time of injection. Non-limiting examples of sensors which would allow determination of the location of the injector include pressure sensors, pH sensors and sensors for dissolved gases, such as oxygen or carbon dioxide. An additional sensor that may be associated with the delivery devices suitable for use with the present invention include sensors which indicate flow of blood such as a backflow port or a backflow lumen which would inform a surgeon or clinician that the needle portion of the delivery device is in an area which has blood flow rather than within a tissue.

While the volume of platelet composition injected may vary based on the size and type of the tissue to be treated, in at least one embodiment, about 1100 μL of platelet composition is injected into the tissue per injection site. In another embodiment, about 200 μL to 2000 μL of the platelet composition is delivered per injection site. In at least one other embodiment, about 100 μL and 10000 μL of the platelet composition is delivered per injection site. In another embodiment, about 50 μL of platelet composition is injected into the tissue per injection site. In one embodiment, the clinician adjusts the injection volume, the number and spacing of injection sites, and the total volume of composition to optimize clinical benefit while minimizing clinical risk.

The total injection volume per treatment may be dose-dependent based on the size of the area to be treated, the size of the injured region of tissue, and/or the size of the area requiring revascularization. In at least one embodiment, the total volume of platelet composition injected into the tissue is as much as can be accommodated by the tissue in a reasonable number of injection sites. In another embodiment, the total volume of composition injected is less than 15000 μL (15 mL). In one embodiment, the volume of platelet composition is customized to what the target tissue can tolerate (with acceptable amounts of acute swelling and pain), and may be as high as 500 milliliters.

The number of injection sites per treatment can be based on the size and shape of the injured region, the desired location of the injections, and the distance separating the injection sites. In at least one embodiment, the number of injection sites can range from 5-25 sites. The distance separating injection sites will vary based on the desired volume of platelet composition to be injected per injection site, the desired total volume to be injected, and the condition of the injured tissue. In at least one embodiment, the distance between injection sites is approximately 2 cm and in at least one other embodiment, the distance between injection sites is 1 cm. In still another embodiment, the separation distance between injection sites can range between about 50 mm and about 2 cm. In another embodiment, the distance between injection sites can be in the range of 0.5 cm to 2.5 cm. In another embodiment, the distance between injection sites is greater than 2.5 cm. Injections can be continuous or interrupted along a needle track instead of as discrete single injections. Continuous or interrupted injections can be delivered as a needle is being advanced into or as it is being withdrawn from the target tissue.

In one embodiment, the platelet composition is injected into the tissue in a pattern that encourages formation of blood vessels. One exemplary pattern is a linear pattern that connects two target areas of tissue so that formation of blood vessels is stimulated along the linear pattern. In another embodiment, the pattern is branched. In particular, the formation of blood vessels comprises the formation of large-bore conduit vessels. In one embodiment, the platelet composition is deposited in close proximity to existing vessels to encourage formation of a functional vascular supply.

FIG. 5 schematically depicts an area of injured tissue after multiple injections of a platelet composition. In one embodiment, the composition is injected into the injured tissue between a first unobstructed blood vessel 404 and a second unobstructed blood vessel 406 parallel 402 to an existing blood vessel. The composition is injected into multiple injection sites 410, 420, 430, 440 and 450 resulting in the diffusion of injectate several millimeters to centimeters from the injection site. The injected composition diffuses such that, if multiple injections are approximately 2 cm apart, the composition forms an overlapping field. For example, composition 412 is injected at injection site 410 and diffuses as depicted in FIG. 5. Further, composition 422 is injected at injection site 420 and diffuses and intermingles with composition 412. This is repeated at injection sites 430, 440 and 450 such that compositions 412, 422, 432, 442 and 452 form a continuous overlapping field. In this embodiment, compositions 412, 422, 432, 442, and 452 are the same composition, in a non-limited example autologous platelet gel. In another embodiment, more than one composition can be injected into a treatment site.

The location of the delivery can vary based on the size and shape of the injured and/or ischemic region of tissue. In at least one embodiment, the composition is delivered only into the injured tissue, while in other embodiments the peri-injury zone around the injured region is treated, and, in at least one other embodiment, the composition is delivered into only the healthy tissue that borders an injured region. In other embodiments, the composition may be delivered to any combination of the regions of injured tissue, tissue in the peri-injury zone, and healthy tissue. The composition may be delivered to healthy tissue within a patient lacking ischemic tissue, for example, to enhance muscle vascularity.

The timing of platelet composition delivery relative to an acute event, such as an acute peripheral vascular obstruction, will be based on the severity of the obstruction, the extent of the ischemia and the condition of the patient. In at least one embodiment, the platelet composition is delivered one to eight hours after revascularization following an obstructive event. In another embodiment, the platelet composition is delivered to the tissue three to four days after obstructive event (after clinical stabilization of the patient, which would make it safe for the patient to undergo a separate procedure). In at least one embodiment, the platelet composition is delivered more than one week after the obstructive event including up to months or years after an obstructive event.

Other times for injecting compositions into tissue are also contemplated, including prior to any obstructive event, prior to the onset of peripheral vascular disease and immediately upon finding an area of ischemic tissue, or after some substantial time period following an ischemic event.

In yet another embodiment, when a subject is suffering from chronic peripheral vascular insufficiency, the platelet composition is administered to the tissue site at any time during the disease course. The platelet composition can be administered early in the course of the disease, such as, but not limited to, when the patient has risk factors for peripheral vascular disease but does not exhibit physical limitations. In another embodiment, the platelet composition is administered late in the disease course, such as, but not limited to, when the patient has limb-threatening ischemia. In another embodiment, the platelet composition is administered when a patient has moderate ischemia, before the onset of limb-threatening ischemia.

In addition to the foregoing uses for the platelet compositions, methods and systems disclosed herein, it will be apparent to those skilled in the art that a variety of injured tissues would benefit from the delivery of a treatment that promotes neovascularization. Examples of such tissues include ischemic tissues in organs or sites including, but not limited to, wounds, gastrointestinal tissue, kidney, liver, skin, muscle, and neural tissue such as brain, spinal cord and nerves.

### EXAMPLES

Experiments have been conducted in laboratory conditions testing the methods and devices of the present invention disclosed herein. These include in vitro studies (described in Examples 1 and 2) in vivo studies conducted in healthy porcine tissue (Examples 3 and 4) and in vivo studies conducted in injured ovine tissue (Example 5).

### Example 1

Various combinations of the components for platelet gel were tested in vitro using human blood, porcine blood, and ovine blood. One composition involved the extraction of 6 mL of platelet rich plasma (PRP) from 60 mL of whole blood (52.5 mL whole blood+7.5 mL anticoagulant [ACD-A, Anticoagulant Citrate Dextrose Solution A, comprising citric acid, sodium citrate and dextrose]). This PRP was combined approximately 10:1 (vol:vol) with bovine thrombin (1000 U/mL stock in 10% CaCl₂), such that mixing occurred only in the targeted tissue. This was the composition tested in vivo as described below.

### Example 2

The ability of fibrinogen to affect the gelling and/or physical properties of platelet gel was directly tested in vitro. PRP and platelet poor plasma (PPP) were prepared from fresh sheep blood using the Medtronic Magellan® Platelet Separator. Autologous fibrinogen was further extracted from the resulting PPP using an ethanol precipitation method. Alternative methods such as cryoprecipitation can be used for isolation of fibrinogen. The precipitated fibrinogen was re-suspended in PRP to generate autologous fibrinogen-fortified PRP (AFFPRP). Two preparations of APG were compared from the same animal—(1) conventional APG made from PRP+1000 U/ml bovine thrombin in a 10:1 ratio and (2) fibrinogen-fortified APG made from AFFPRP+1000 U/ml bovine thrombin in a 10:1 ratio. The fibrinogen-fortified APG was noticeably firmer/harder than the conventional APG generated from the same animal's blood. This confirms the utility of fibrinogen to augment the mechanical properties of APG without reducing the gelling rate.

### Example 3

It has been successfully demonstrated that intramuscular delivery of APG as two separate components (autologous PRP and bovine thrombin) that meet and clot in the tissue can be safely achieved in vivo.

Model & Access: A healthy pig model was used to test the safety and efficacy of delivery into cardiac muscle tissue. One hundred and eighty milliliters of unheparinized blood was obtained and used to make 18 cc of PRP using a Medtronic Magellan® Autologous Platelet Separator on the day of the procedure. The animal was then heparinized to an activated clotting time (ACT) in the 250-300 range. A median sternotomy provided access to the epicardial surface of the heart.

Injections: Three injection systems were tested: System 1, a 27 gauge syringe to deliver PRP alone; System 2, an 18 gauge stainless steel needle containing a 2-lumen beveled catheter (0.0085-inch internal diameter [ID] each) with luer-lock into the needle and two independent proximal syringes (12 mL and 1 mL in size). The syringes were operated using a one-handed manifold which ensured simultaneous injection of the two components at the desired ratio (in this example, approximately 11:1). This was used to inject autologous PRP and bovine thrombin; and System 3, a suction injector which combined a suction head (to be placed on the epicardial surface of the heart) with a dual-needle injector. The suction member is driven by a vacuum pump which achieves local stabilization of the beating heart. It additionally draws the cardiac wall up into the suction cup so that the needles (entering the tissue parallel to the plane of the chamber) can be delivered at a controllable depth. The needles are driven by two separate syringes, also anchored to a one-handed injection manifold as described above. A 12 mL and 1 mL syringe were used to ensure delivery of the desired ratio of autologous PRP and bovine thrombin (in this example 11:1).

Multiple injections of small volume (200-400 μl/each) were performed via an epicardial surgical approach. For injections using Systems 1 and 2 above, injections were made perpendicular to the target tissue, and a “depth stop” was used to ensure injection to a desired depth. Target depth was 5 mm in the left ventricle and 3 mm in the right ventricle. The depth-stop consisted of a C-shaped member with a central hole through which the injection needle was passed. A side-screw (which narrows the lumen size of the depth-stop as it is screwed in) was used to anchor the depth-stop along the outside of the needle at the desired position along its length. As the needle is gently advanced into the target tissue by the application of a force, the needle reaches the level of the depth-stop, beyond which it could not be advanced. Thus, this system ensures a fixed depth of needle penetration into tissue and ensures intramural injection occurs when wall thickness is known or estimatable.

For all injections in this study, the Medtronic Starfish® cardiac stabilizer (available from Medtronic, Inc., Minneapolis, Minn. USA) was used to provide procedural stabilization of the beating heart.

Target Tissue: Injections were performed in the left ventricle (LV, at its base, mid-position, and apex) and right ventricle (RV, at its base, mid-position, and apex). Injections into the LV were targeted to a 5 mm depth. Injections into the RV were targeted to a 3 mm depth.

Compositions: Different injectates were tested.

1) autologous PRP alone—to determine whether clot formation occurs in absence of exogenous thrombin

2) autologous PRP+bovine thrombin

3) Each of the above injections was performed with and without addition of toluidine blue dye to the autologous PRP. This was to test the utility and efficacy of a tracking dye for experimental purposes.

4) Saline control

Results: Hemostasis after APG injections was excellent. Specifically, multiple left ventricular injections of up to 1000 μl/each of APG (PRP:thrombin at 10:1) into healthy porcine myocardium were feasible and clinically safe. No adverse events were observed for up to 3 days of follow-up. Multiple right ventricular injections of up to 200 μl/each of APG (PRP:thrombin at 10:1) into healthy porcine myocardium were feasible and clinically safe. No adverse events were observed over a 2 hour follow-up period.

Twenty-three injections were well-tolerated without arrhythmia, hypoxemia, or any clinical compromise during or for 1 hr following the last injection. No thrombotic or thromboembolic sequellae were found post-mortem. All 23 injections were successful, and injection sites examined during necropsy.

Platelet gel can be formed from PRP alone without the addition of exogenous thrombin. Platelet rich plasma injected into myocardium alone (without thrombin) surprisingly gels in situ. The present inventor has formulated the non-binding hypothesis that tissue thrombin may be present in sufficient quantities to trigger this gelling reaction. Therefore, PRP may be used to create APG within the tissue when injected alone into myocardium in vivo.

### Example 4

Platelet rich plasma can be tracked in tissue by adding toluidine blue dye to the PRP. This dye does not noticably change the gelling characteristics (rate of gelling, extent of gelling, firmness of resultant gel) of PRP upon its combination with thrombin.

The pattern of APG distribution upon injection into myocardium was evaluated in vivo. In three pigs, injections of APG labeled with toluidine blue demonstrated that each injection results in distribution of the APG in all directions within the tissue. The greatest spread is along the plane of the ventricle. APG travels radially in the plane of the ventricle up to 1.5 cm. In some injections, APG was detected more than 1.5 cm away from the injection site. It is likely that APG travels during the gelling process until enough gelling has occurred to prohibit further spread of the material within the tissue.

### Example 5

The acute effects of APG injection into ischemic myocardium were studied in a sheep anterior infarct model. In this model, myocardial infarction results in deleterious structural and functional changes that occur within minutes of the injury. The early hallmarks of remodeling include ventricular dilatation, wall thinning, akinesis and often dyskinesis. Over time, these changes progress as remodeling continues. It was determined that early intervention post-infarction by providing APG to the injured myocardium can stunt this remodeling process. Such APG treatment resulted in a striking and significant increase in neovascularization of infarcted cardiac muscle tissue above control infarcted animals not receiving APG.

The experiments indicated that injections were safe and well tolerated when made into infarct or non-infarct tissue, and that they can be performed safely as early as 1 hr post-MI. Controlled injections were possible with or without a cardiac stabilization device, and it was possible to make the injections without exogenous cardiac pacing. Injections were made both orthogonally and obliquely to the cardiac muscle surface at intervals of 0.5 to 2.5 cm. The total injectate volume was tested to be safe at as high as 15.0 mL per heart, and the volume of individual injections as high as 1100 μl per injection site.

In twelve sheep receiving APG one hour after infarction and followed for 8 weeks, APG was surprisingly associated with neovascularization in the target ischemic tissue. This effect was not expected because the target tissue is, by definition, ischemic, and provides a poor environment for cells to survive, let alone grow to generate functional structures. In twelve of twelve animals, a striking number of vessels were observed within the APG-treated infarct region at 8 weeks (FIGS. 10 and 11). Red blood cells observed within the blood vessels are highly suggestive of perfusion, implicating that these are functional vessels capable of providing fresh blood supply to surrounding tissue, rather than merely empty tufts of blood vessels. This suggests that APG (even when delivered in a diffuse fashion) is capable of generating normal, well-differentiated blood vessels, and targeting their growth to ensure perfusion, presumably by nearby supplier vessels. That APG contains the full complement of signalling, targeting, and neovascularization-promoting activities was unexpected. A striking and statistically significant increase in vascularity was observed in APG-treated infarct tissue compared with animals experiencing infarction without APG injection.

The experiments revealed that there is animal-to-animal (and presumably patient-to-patient) variability in clotting rate of APG, and (to a lesser degree) the mechanical properties of APG. Methods that demonstrate improved APG clotting rate/strength include using high-dose bovine thrombin at 1000 U/mL to make APG, and using cooled (˜0° C.) thrombin to make APG. Additionally the clotting rate/strength can be improved by fortifying autologous PRP with concentrated fibrinogen (e.g., autologous fibrinogen prepared by ethanol extraction or frozen preparation). Also, the post injection clotting rate/strength can be improved by extremely careful handling of PRP prior to injection to ensure minimal pre-activation.

Several methods were identified to enhance retention of the injectate in the target tissue and to address possible leakage/backbleed issues. These methods include using high-dose bovine thrombin at 1000 U/mL to make APG. An agitator mechanism can be used in the PRP delivery and/or refill chamber to prevent settling or dissolution of the PRP. This will ensure delivery of a homogeneous PRP to the target tissue and facilitate improved clotting. Other methods include allowing the needle to dwell for 5-10 second in the injection site after the injectate has been delivered, using an oblique angle to lengthen the injection track in the tissue, and local stabilization of the injection site on entry of the needle (to prevent tearing). Each of these methods was tested in the aforementioned Examples.

Using cooled (˜0° C.) thrombin to make APG also enhances retention of the injectate in the target tissue. For the embodiment using cooled thrombin, a refrigerated/cooled chamber can be used in the thrombin delivery and/or refill chamber.

The injected compositions can be visualized by intra-operative ultrasound, which can be used to confirm adequate needle placement and retention. The ultrasound can be from a separate device or can be included within the delivery system (e.g. similar to intravascular ultrasound [IVUS]). Additionally, intra-operative visualization can be obtained by surface or intravascular ultrasound for peripheral tissues such as limb muscle.

Unintended delivery into the blood space (in this experimental example, the cardiac chamber or epicardial vessels) can be avoided by using imaging guidance during injections, such as that provided by ultrasound or IVUS. Additionally, it was found that direct epicardial injections into the apex of the heart should be avoided to prevent chamber puncture. Instead, oblique injections should be used to access apical tissue. Also, a device can be used to inform the operator when the delivery portion of the delivery device is in an undesired position for delivery, such as in the ventricle or in a neighboring vessel. Such a device may have at least one sensor include, but not limited to, a pressure sensor, a color detector, an oxygen sensor, a carbon dioxide sensor or a lumen to express backflowing blood under pressure that generates a unique signal when the delivery system is positioned such that its target is in a blood space. Once alerted, the user can re-position the device before delivering the composition.

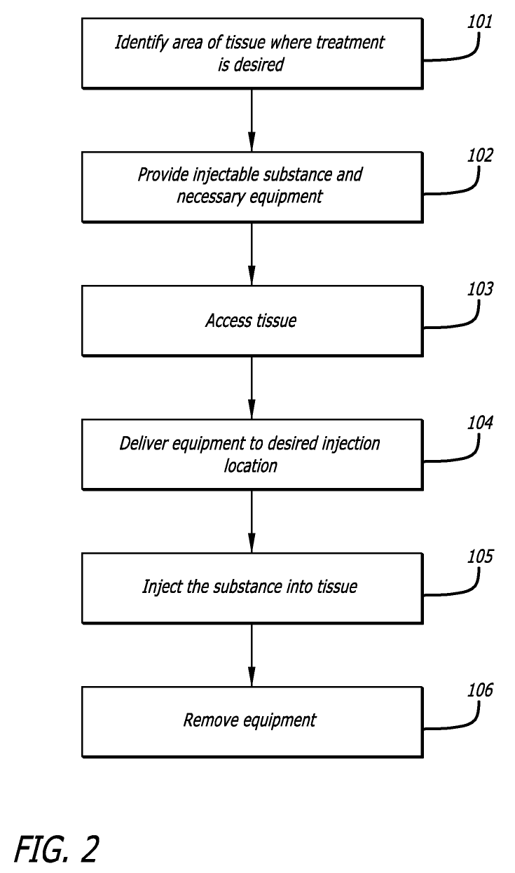

The current invention discloses a method of treating ischemic tissue by injecting substances that promote neovascularization. Referring to FIG. 2, the method generally comprises the steps of identifying and/or imaging the ischemic region where therapy is desired 101, determining an appropriate substance for injecting into the tissue to achieve the desired effect (neovascularization), selecting the appropriate device for injecting the substance into the tissue 102, accessing the tissue 103, delivering the substance and delivery device to the desired treatment location 104, injecting the substance into the tissue 105 and withdrawing the device 106. The method and devices for injecting the composition (substance/injectate), the composition, and the processes for delivery have been discussed herein.

### Example 6

A rabbit hindlimb ischemia model is created according to the methods of Niagara et al. (J. Vasc. Surg. 40:774-85, 2004). Briefly, the animals are randomized to a treatment and control groups and anesthetized with xylaxine/ketamine. A longitudinal incision is made in the left hindlimb, extending inferiorly from the inguinal ligament to a point just proximal to the patella. The left femoral artery is dissected free from the surrounding tissues, and the femoral vein along its entire length, and all the branches of the femoral artery (including the inferior epigastric, deep femoral, lateral circumflex, and superficial epigastric arteries) are also dissected. After dissection of the popliteal and saphenous arteries distally, the external iliac artery and all of the above arteries are ligated. Then, the left femoral artery is completely resected from its origin at the external iliac artery to the point where it bifurcates to form the saphenous and popliteal arteries. The femoral vein is left in situ. Excision of the femoral artery results in retrograde thrombosis and occlusion of the external iliac artery. The incision is closed and the rabbits are allowed to recover for 10 days.

Platelet rich plasma and thrombin, in a variety of ratios including 10:1, are then injected into the ischemic tissue using several approaches. In group one, a two-lumen injection system such as, but not limited to, the injection system of FIG. 3, is used to directly inject the composition into the ischemic tissue (e.g. FIG. 7). In group two, a transvascular venous approach is used to access vein 12 (FIG. 1) with an injection catheter and just beyond the point of occlusion, a needle from the catheter pierces the vein and passes into the ischemic tissue. In group three, a transvascular arterial approach is used to access artery 10 (FIG. 1) with an injection catheter in which a needle perforates the artery near the point of occlusion to enter into the ischemic tissue. For groups one, two, and three, the needle may be guided by ultrasound or fluoroscopic means. The needle may be guided some distance to a point at the distal end of the ischemic tissue and the composition injected while retracting the needle, leaving a track of composition in the void created by the passage of the needle. The needle and injection catheter are then completely retracted.

The animals are survived chronically and monitored for 8-16 weeks. Non-destructive follow-up determinations of functional revascularization (such as limb blood pressures, peripheral arteriography, and peripheral venography) are performed to assess the in-growth and functionality of the vascular supply at different timepoints following therapy. At the end of the follow-up period, the animals are sacrificed and histologic examination of the tissues for neovascularization is performed.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosed invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the elements otherwise claimed. No language in the specification should be construed as indicating any non-claimed element essential to the practice of the methods disclosed herein.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of the invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for neovascularization of tissue comprising:
a platelet composition and
at least one delivery device for introducing said platelet composition into said tissue;
wherein said platelet composition induces neovascularization in said tissue.

2. The system of claim 1 wherein said platelet composition is selected from the group consisting of platelet gel, platelet rich plasma and platelet poor plasma.

3. The system of claim 1 wherein said platelet composition is autologous.

4. The system of claim 2 wherein said platelet gel is formed from platelet poor plasma or platelet rich plasma and an activating agent.

5. The system of claim 4 wherein said activating agent is thrombin.

6. The system of claim 5 wherein said platelet gel is formed from platelet rich plasma or platelet poor plasma and thrombin at a ratio of between about 5:1 and about 25:1.

7. The system of claim 2 wherein said platelet composition comprises platelet rich plasma without an exogenous source of thrombin.

8. The system of claim 1 wherein said platelet composition further comprises a bioactive agent.

9. The system of claim 8 wherein said bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, cells and combinations thereof.

10. The system of claim 1 wherein said platelet composition further comprises a contrast agent.

11. A method of inducing neovascularization in tissue comprising:
providing a platelet composition at a treatment site in said tissue wherein said platelet composition induces neovascularization in said tissue.

12. The method according to claim 11 wherein said platelet composition is selected from the group consisting of platelet gel, platelet rich plasma and platelet poor plasma.

13. The method according to claim 11 wherein said platelet composition is autologous.

14. The method of claim 1 wherein said delivery device is an injection catheter.

15. The method of claim 14 wherein said delivery device introduces said platelet composition to said treatment site through a route selected from the group consisting of a trans-arterial approach, a trans-venous approach and a trans-cutaneous approach.

16. The method of claim 14 wherein said introduction of said platelet composition to said treatment site is performed with in situ visualization.

17. The method of claim 1 wherein said platelet composition is introduced to said treatment site at multiple injection sites along a path.

18. The method of claim 1 wherein said treatment site is selected from the group consisting of the ischemic area, the peri-ischemic area and the healthy tissue surrounding the ischemic area.

19. A method of treating peripheral vascular disease comprising:
providing a platelet composition into a treatment site in ischemic tissue wherein said composition induces neovascularization of said tissue; and
injecting a cell preparation into said tissue.

20. The method of claim 19 wherein said platelet composition is selected from the group consisting of platelet gel, platelet rich plasma and platelet poor plasma.

21. The method according to claim 19 wherein said platelet composition is autologous.

22. The method according to claim 20 wherein said platelet gel is formed from platelet poor plasma or platelet rich plasma and an activating agent.

23. The method according to claim 22 wherein said activating agent is thrombin.

24. The method according to claim 20 wherein said platelet composition comprises platelet rich plasma without an exogenous source of thrombin.

25. The method according to claim 19 wherein said platelet composition further comprises a bioactive agent selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

26. The method according to claim 19 wherein said cell preparation comprises cells of one or more cell types selected from the group consisting of somatic, germ-line, fetal, embryonic, post-natal cells and adult cells.

27. The method according to claim 26 wherein said cell preparation comprises cells isolated from one or more tissue types selected from the group consisting of adipose, brain, muscle, endothelial, blood, bone marrow, heart, testes and ovaries.

28. The method according to claim 27 wherein said cells are autologous.

29. The method according to claim 27 wherein said cells are modified prior to implantation.

30. The method according to claim 19 wherein said cell preparation is provided to said tissue after neovascularization is initiated in said tissue.

31. The method according to claim 19 wherein said platelet composition and said cell preparation are provided to said tissue approximately simultaneously.

32. The method according to claim 19 wherein said treatment site is selected from the group consisting of the ischemic area, the peri-ischemic area and the healthy tissue surrounding the ischemic area.

33. A system for neovascularization of tissue comprising:
thrombin and at least one delivery device for introducing said thrombin into said tissue wherein thrombin induces neovascularization in said tissue.
